(19) United States
(12) Patent Application Publication
(10) Pub. No.: US 2014/0275139 A1
(43) Pub. Date: Sep. 18, 2014

(54) MDR METHOD AND PRODUCTS FOR TREATING HIV/AIDS

(71) Applicant: HIV Diagnostics, Inc., Lexingtown, KY (US)

(72) Inventor: Knox Van Dyke, Morgantown, WV (US)

(73) Assignee: HIV Diagnostics, Inc., Lexingtown, KY (US)

(21) Appl. No.: 14/206,266

(22) Filed: Mar. 12, 2014

Related U.S. Application Data
(60) Provisional application No. 61/777,380, filed on Mar. 12, 2013.

(51) Int. Cl. A61K 31/4745; A61K 45/06

(52) U.S. Cl. CPC A61K 31/4745; A61K 45/06
USPC 514/279

(57) ABSTRACT

Multidrug resistance reversers of the d-tetrandrine family are used concurrently with protease inhibitors to treat HIV/AIDS.

### CROSS REFERENCE TO RELATED APPLICATIONS

The present application claims the benefit of U.S. Provisional Patent Application No. 61/777,380, entitled NEW COMBINATION TREATMENT FOR HIV OR AIDS, filed on Mar. 12, 2013, the entire contents of which are incorporated by reference.

### FIELD AND BACKGROUND

The present invention relates to the treatment of HIV or AIDS. Protease inhibitors are a class of antiviral drugs that are widely used to treat HIV/AIDS. Protease inhibitors prevent viral replication by selectively binding to viral proteases, such as HIV-1 protease. This blocks proteolytic cleavage of protein precursors that are necessary for the production of infectious viral particles.

### SUMMARY OF THE INVENTION

In the present invention, HIV/AIDS is treated by the concurrent administration of at least one protease inhibitor and at least one multi-drug resistance inhibitor. Among other benefits, this invention is effective in treating HIV/AIDS infections of the brain.

### DESCRIPTION OF THE PREFERRED EMBODIMENTS

HIV/AIDS infection of the brain is particularly nasty, and difficult to treat. When HIV invades the brain, it can cause premature dementia and other central nervous system disorders. It is difficult to treat because of the blood brain barrier, which keeps many drugs from entering the brain. At least eight HIV protease inhibitors are known (as listed below in Table 1) and there are many more to be developed. These drugs are effective in peripheral sites in the body but are not effective when the virus is in the brain. Protease inhibitors do not penetrate the blood-brain barrier.

TABLE 1

1. Amprenavir
2. Indinavir
3. Nelfinavir
4. Saquinavir
5. Ritonavir
6. Fosamprenavir
7. Tipranavir
8. Atazanavir

These are all injectable drugs.

It is believed that at least one mechanism by which the blood brain barrier rejects otherwise helpful drugs from crossing the barrier is the P-glycoprotein pump (PGP) at blood tissue barriers. See Fromm, Trends in Pharmacological Sciences—TIPS 25, #8, 423-429, 2004. Basically PGP acts as an energy driven (ATP-dependent) pump which exists in various organs and endothelial cells which line the blood carrying capillaries that form the blood brain barrier. The pump is anatomically arranged so that it keeps many drugs from entering the brain and acts as a barrier to many important therapeutic drugs.

By concurrently administering an MDR inhibitor with a protease inhibitor, we overcome the resistance posed by the PGP pumps. While not wishing to be bound to any particular theory of action, I believe that MDR inhibitors inhibit the PGP or PGP-like pumps by inhibiting the utilization of ATP (adenosine triphosphate). This causes the drug extruding action of the pump at the blood brain barrier to stop. With the pumps turned off, the protease inhibitors will penetrate the brain, as seen in mouse knockout models of PGP.

The MDR inhibitor should inhibit the MDR (Multiple Drug Resistance) pump without adding important toxicity. Preferably, it shouldn't cause an increase in the metabolism of the protease inhibitors. It is preferably orally active and preferably has a respectable half-life of a day or even multiple days.

I have found that a variety of natural and synthetic bisbenzyl isoquinolines effectively inhibit the multiple drug resistant (MDR) mechanism which is present in cancer cells, malarial parasites, T and B lymphocytes, and the blood brain barrier. See U.S. Pat. Nos. 5,025,020; 5,332,747; 6,528,519; 6,911,454; 6,124,315 and 6,962,927. The genetic sequence that codes for the MDR protein is very similar in all four cases.

The d-tetrandrine family member of the following structural formula are preferable MDR inhibitors:

[chemical structure]

where R₁ and R₁′ are the same or different short chained carbon based ligand including without limitation, CH₃, CO₂CH₃ or H; and R₂ is CH₃ or C₂H₅; and R₃ is CH₃ or hydrogen; and where the chemical structure has the “S” isomeric configuration at the C-1′ chiral carbon location.

The preferred members of the d-tetrandrine family include the following representative examples, which are not intended to be exhaustive: d-tetrandrine, isotetradine, hernandezine, berbamine, pyenamine, phaeanthine, obamegine, ethyl fangchinoline and fangchinoline. In all of these examples, R₁ and R₁′ constitute the methyl group. Variation within group occurs in that R₂ and R₃ may constitute either a methyl group or hydrogen, and the isometric configuration of the compounds at the C-1′ and C-1′ chiral carbon positions is either R (rectus) or S (sinister). The rules for R and S configuration can be found in Morrison and Boyd, Organic Chemistry, ^{4th} Edition, copyright 1983 by Allyn and Bacon, at pp. 138-141. As noted above, the chiral configuration at C-1′ is “S” for members of the d-tetrandrine family. In addition, hernandezine includes a methoxy group at the C-5 position.

The most preferred member of the claimed tetrandrine family is d-tetrandrine. Methods for extracting and/or purifying d-tetrandrine are disclosed in U.S. Pat. No. 6,218,541 and in Published Patent Application No. 2011/0105755.

The term concurrent administration as used herein refers to the administration of the drugs either simultaneously or sufficiently close together that therapeutic levels of both are present in the bloodstream, and especially at the blood-brain barrier, at the same time. The dose and timing for administration of the particular protease inhibitor to be used is determined by reference to a standard Physician's Desk Reference. The timing of administration of the MDR inhibitor is tuned to correspond to the timing of administration of the protease inhibitor.

The d-tetrandrine family member and the protease inhibitor can be formulated together into a single formula, they can be formulated separately and administered either simultaneously or sufficiently close together that the blood-brain barrier is exposed to both simultaneously. The two drugs formulated separately may be sold as part of a “kit”. The usage ratio of the d-tetrandrine family member to a protease inhibitor will vary from patient to patient and as a function of the protease inhibitor used, within a range of from about 0.04 to about 170, more typically from about 1 to 100.

It is believed that the optimum dosage procedure would be to administer the d-tetrandrine family multidrug resistance reverser in oral doses of from about 50 to about 1000 mg per square meter per day, more preferably 250-700, and most preferably about 500, (probably in two to four doses per day) over a period of from about 4 to about 14 days. The dosage level for the d-tetrandrine family member will vary from case to case, based on the patient and on the protease inhibitor used. The protease inhibitor is then administered at usual dosage levels (possibly somewhat less in view of the potentiation effect of the resistance reverser) once or more during the course of the resistance reverser dosing. For example, during a four day period of d-tetrandrine administration, the protease inhibitor would be administered on the beginning of the third day. Over a 14 day period, the protease inhibitor or drugs might be administered on day 5 and day 10, or on days 4, 8 and 12.

The d-tetrandrine family bisbenzylisoquinolines have two nitrogen locations and hence can exist in the free base form or as a mono or di-acid salt. Because of the enhanced solubility of the salt form of pharmaceutical ingredients, the salt forms are used in formulating pharmaceutical compositions. The active ingredient thus solubilizes more quickly and enters the bloodstream faster. The free base form is not soluble in water. However, it has recently been surprisingly found by a co-worker that the free base formulations of d-tetrandrine family members are absorbed into the bloodstream substantially as rapidly as formulations of the di-acid salt members of the family. Accordingly, we propose to use either the free base or the di-acid salt of the d-tetrandrine family member in our protease inhibitor—MDR inhibitor formulations.

The preferred formulations comprise a member of the d-tertrandrine family combined with a suitable pharmaceutical carrier. The pharmaceutical carrier can be a liquid or a solid composition. A liquid carrier will preferably comprise water, possibly with additional ingredients such as 0.25% carboxymethylcellulose. The solid carrier or diluent used may be pregelatinized starch, microcrystalline cellulose or the like. It may also be formulated with other ingredients, such as colloidal silicon dioxide, sodium lauryl sulfate and magnesium stearate.

A 200 mg capsule, tablet or liquid dosage formulation is most preferred. The most preferred dose of about 500 mg/square meter/day is roughly 1000 mg per day for a 190 pound patient six feet tall. Such a patient can fulfill the dosage requirements by taking five capsules during the course of the day, for example three in the morning and two in the evening, or one at a time spaced out over the day. A smaller person weighing 125 pounds at a height of five feet six inches would require four 200 mg capsules during the course of the day.

Of course, it is understood that the above disclose some embodiments of the invention, and that various changes and alterations can be made without departing from the scope of the invention as set forth in the attached claims and equivalents thereof.

1. A method of treating HIV/AIDS comprising: concurrently to a patient affected with HIV/AIDS a protease inhibitor and an MDR inhibitor.

2. The method of claim 1 in which the MDR inhibitor is a member of the d-tetrandrine family having the following structural formula: [chemical structure]
where R₁ and R₁′ are the same or different short chained carbon based ligand including without limitation, CH₃, CO₂CH₃ or H; and R₂ is CH₃ or C₂H₅; and R₃ is CH₃ or hydrogen, has the “S” isomeric configuration at the C-1′ chiral carbon location.

3. The method of claim 2 wherein said member of the d-tetrandrine family is selected from the group consisting of: d-tetrandrine, isotetrandine, hernandezine, berbamine, pyenamine, phaeanthine, obamegine, ethyl fangchinoline and fangchinoline.

4. The method of claim 3 wherein said member of the d-tetrandrine family is d-tetrandrine.

5. The method of claim 3 in which the d-tetrandrine family member and the protease inhibitor are formulated together into a single formula.

6. The method of claim 3 in which the d-tetrandrine family member and the protease inhibitor are formulated separately and administered either simultaneously or sufficiently close together that the HIV/AIDS is exposed to both simultaneously.

7. The method of claim 3 in which the d-tetrandrine family member and protease inhibitor are administered in a usage ratio of d-tetrandrine family member to protease inhibitor, within a range of from about 0.04 to about 170.

8. The method of claim 3 in which the d-tetrandrine family member and protease inhibitor are administered in a usage ratio of d-tetrandrine family member to protease inhibitor, within a range of from about 1 to 100.

9. The method of claim 3 in which the d-tetrandrine family is administered in oral doses of from about 50 to about 1000 mg per square meter per day over a period of from about 4 to about 14 days, and the protease inhibitor is then administered at usual dosage levels once or more during said 4 to 14 days.

10. The method of claim 3 in which the d-tetrandrine family is administered in oral doses of from about 250-700 mg per square meter per day over said period of from about 4 to about 14 days.

11. The method of claim 3 in which the d-tetrandrine family is administered in oral doses of about 500 mg per square meter per day over said period of from about 4 to about 14 days, in two to four doses per day.

12. The method of claim 3 in which the HIV/AIDS is in the brain.

13. The method of claim 1 in which the HIV/AIDS is in the brain.

14. The method of claim 1 in which the MDR inhibitor and the protease inhibitor are formulated together into a single formula.

15. The method of claim 14 in which the HIV/AIDS is in the brain.

16. The method of claim 1 in which the MDR inhibitor and the protease inhibitor are formulated separately and administered either simultaneously or sufficiently close together that the HIV/AIDS is exposed to both simultaneously.

17. The method of claim 17 in which the HIV/AIDS is in the brain.

18. A pharmaceutical composition comprising a protease inhibitor combined with an MDR inhibitor.

19. The pharmaceutical composition of claim 18 in which said MDR inhibitor is a member of the d-tetrandrine family having the following structural formula: [chemical structure]
where R₁ and R₁′ are the same or different short chained carbon based ligand including without limitation, CH₃, CO₂CH₃ or H; and R₂ is CH₃ or C₂H₅; and R₃ is CH₃ or hydrogen, has the “S” isomeric configuration at the C-1′ chiral carbon location.

20. A pharmaceutical kit including protease inhibitor, and an MDR inhibitor.

21. The kit of claim 20, is which the MDR inhibitor is a formulation comprising a member of the d-tetrandrine family having the following structural formula: [chemical structure]
where R₁ and R₁′ are the same or different short chained carbon based ligand including without limitation, CH₃, CO₂CH₃ or H; and R₂ is CH₃ or C₂H₅; and R₃ is CH₃ or hydrogen, has the “S” isomeric configuration at the C-1′ chiral carbon location.
